(19) United States
(12) Patent Application Publication
(10) Pub. No.: US 2024/0307602 A1
(43) Pub. Date: Sep. 19, 2024

(54) CANNULATION SYSTEM

(71) Applicant: Paul Zamudio, Dobbs Ferry, NY (US)

(72) Inventor: Paul Zamudio, Dobbs Ferry, NY (US)

(21) Appl. No.: 18/606,166

(22) Filed: Mar. 15, 2024

Related U.S. Application Data
(60) Provisional application No. 63/452,265, filed on Mar. 15, 2023.

(51) Int. Cl. A61M 1/36; A61M 25/06; A61M 39/02

(52) U.S. Cl. CPC A61M 1/3659; A61M 25/0631; A61M 25/0637; A61M 39/0247

(57) ABSTRACT

A system comprises a cannula and a needle, with the cannula and needle coaxially arranged, a needle retraction assembly configured to retract the needle, and a safety enclosure wherein the needle retraction assembly is configured to draw the needle out of the cannula and into the safety enclosure.

### CROSS REFERENCE TO RELATED PATENT APPLICATIONS

This patent application, also claims the priority and benefit under 35 U.S.C. § 119(e) of U.S. Provisional Patent Application Ser. No. 63/452,265 filed Mar. 15, 2023, entitled “CANNULATION SYSTEM”. U.S. Provisional Patent Application Ser. No. 63/452,265 is herein incorporated by reference in its entirety.

### TECHNICAL FIELD

Embodiments disclosed herein are generally related to medical devices. Embodiments are additionally related to cannulation. Embodiments are also related to tools for hemodialysis. Embodiments are further related to fistulas. Embodiments are further related to catheters. Embodiments are also related to systems, methods and apparatuses for improved fistula cannulas and hemodialysis needle systems.

### BACKGROUND

Chronic kidney disease affects one in seven adults in the United States. This constitutes approximately 37 million Americans. The risks are even higher for those with chronic conditions such as diabetes or high blood pressure. Of this group, approximately 800,000 suffer from end stage renal disease. 70% of these patients are on dialysis.

Hemodialysis is used to purify the blood of a person whose kidneys are not functioning properly. There are 2 types of hemodialysis—in center hemodialysis and home hemodialysis. Both procedures require vascular access.

There are three common methods of vascular access used for hemodialysis: a tunneled venous catheter, an arteriovenous fistula, and a synthetic graft. There are various advantages to each of these vascular access tools. However, with all such vascular access tools there is significant risk of damage to the patient's dialysis access and innate vasculature.

For example, current dialysis needles are sharp and are generally of a large gauge. These characteristics create moderate to high risk of damage to the patient's vascular access and surrounding tissues. In addition, accurate and precise placement of standard needles is heavily dependent on the provider or patient's proficiency and comfort level in placing the needles.

Furthermore, cannulation is an exceedingly common medical procedure. Every cannulation procedure performed by a medical provider exposes them to the possibility of an unintentional needle stick. Needle sticks compromise the health and safety of medical providers, and in aggregate, present a serious risk to medical providers.

As such, there is a need in the art for improved cannulation systems as disclosed in the embodiments detailed herein.

### SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, an aspect of the disclosed embodiments to provide medical devices.

In addition, it is another aspect of the embodiments to provide vascular access devices.

It is another aspect of the disclosed embodiments to provide methods and systems for hemodialysis.

It is another aspect of the disclosed embodiments to provide methods and systems comprising fistulas or catheters.

It is another aspect of the disclosed embodiments to provide improved methods, systems, and apparatuses for cannulation.

The aforementioned aspects and other objectives and advantages can now be achieved as described herein. In one embodiment, a system comprises a cannula, a needle, a needle retraction assembly, and a safety enclosure wherein the needle retraction assembly is configured to draw the needle out of the cannula and into the safety enclosure. In an embodiment, the needle is configured to be inside, and coaxial, with the cannula.

In an embodiment, the system further comprises at least one support wing attached to the needle retraction assembly. The support wing further comprises an adhesive and a removable cover covering the adhesive. In an embodiment, the support wing further comprises a pre-formed slit along the support wing.

In an embodiment, the safety enclosure is configured to be disengaged from the needle retraction assembly.

In an embodiment, the system further comprises a connection port. The connection port can further comprise a Luer lock system configured on the needle retraction assembly.

In an embodiment, the needle retraction assembly further comprises a spring engaged to the needle with a spring stop, a housing, and a button formed on the housing, the button being used to release the spring to retract the needle.

In an embodiment, the cannulation system comprises a needle configured coaxially inside a cannula, a needle retraction assembly comprising a spring engaged to the needle with a spring stop and a button formed on the housing, the button used to release the spring and a safety enclosure wherein the needle retraction assembly is configured to draw the needle out of the cannula and into the safety enclosure when the button is operated. In an embodiment, the cannulation system further comprises at least one support wing attached to the needle retraction assembly. In an embodiment, the support wing further comprises an adhesive and a removable cover covering the adhesive. In an embodiment, the safety enclosure is configured to be disengaged from the needle retraction assembly. In an embodiment, the cannulation system further comprises a Luer lock system configured on the needle retraction assembly.

In another embodiment, a system comprises a needle configured coaxially inside a cannula, a needle retraction assembly, a safety enclosure wherein the needle retraction assembly is configured to draw the needle out of the cannula and into the safety enclosure, and tubing connected to the cannula. In an embodiment the system further comprises a joint connecting the tubing to the cannula. In an embodiment, the system further comprises a chamber connecting the cannula to the tubing, wherein fluid can flow from the cannula through the chamber into the tubing. In an embodiment, the system further comprises at least one support wing attached to the needle retraction assembly, the support wing further comprising: an adhesive, a removable cover covering the adhesive, and a preformed slit along the support wing. In an embodiment, the system further comprises a Luer lock system configured on the needle retraction assembly. In an embodiment, the needle retraction assembly further comprises a spring engaged to the needle with a spring stop, a housing, and a button formed on the housing, the button used to release the spring.

### BRIEF DESCRIPTION OF THE FIGURES

The accompanying figures, in which like reference numerals refer to identical or functionally similar elements throughout the separate views and which are incorporated in, and form a part of the specification, further illustrate the embodiments and, together with the detailed description, serve to explain the embodiments disclosed herein.

FIG. 1A illustrates a cannulation system, in accordance with the disclosed embodiments;

FIG. 1B illustrates a top view of a cannulation system, in accordance with the disclosed embodiments;

FIG. 1C illustrates aspect of the wings associated with the cannulation system, in accordance with the disclosed embodiments;

FIG. 1D illustrates aspects of the cannulation system, in accordance with the disclosed embodiments;

FIG. 1E illustrates aspects of the cannulation system and connection to tubing, in accordance with the disclosed embodiments;

FIG. 1F illustrates a side view of the aspects of the cannulation system, in accordance with the disclosed embodiments;

FIG. 1G illustrates aspects of a double lumen cannulation system, in accordance with the disclosed embodiments;

FIG. 1H illustrates aspects of a double lumen cannulation system, in accordance with the disclosed embodiments;

FIG. 1I illustrates aspects of a triple lumen cannulation system, in accordance with the disclosed embodiments;

FIG. 2A illustrates a needle with a spring in an extended configuration, in accordance with the disclosed embodiments;

FIG. 2B illustrates a head on view of a spring stop in accordance with the disclosed embodiments;

FIG. 2C illustrates a needle with a spring in a compressed configuration, in accordance with the disclosed embodiments;

FIG. 3A illustrates a needle retraction assembly, in accordance with the disclosed embodiments;

FIG. 3B illustrates another view of a needle retraction assembly, in accordance with the disclosed embodiments;

FIG. 4 illustrates a needle retraction assembly and a safety enclosure, in accordance with the disclosed embodiments;

FIG. 5A illustrates aspects of a cannulation system, in accordance with the disclosed embodiments;

FIG. 6A illustrates a needle retraction assembly with the needle in the non-deployed position, in accordance with the disclosed embodiments;

FIG. 6B illustrates a needle retraction assembly with the needle in the deployed position, in accordance with the disclosed embodiments;

FIG. 6C illustrates a cross-sectional view of needle retraction assembly/flash chamber area, in accordance with the disclosed embodiments;

FIG. 6D illustrates a cross-sectional view of needle retraction assembly undeployed and deployed respectfully, in accordance with the disclosed embodiments;

FIG. 7A illustrates the system being inserted in a patient to achieve vascular access, in accordance with the disclosed embodiments;

FIG. 7B illustrates the system inserted in a patient with the needle retracted, in accordance with the disclosed embodiments; and

FIG. 8 depicts a flow chart of steps associated with a method of using a cannulation system, in accordance with the disclosed embodiments.

### DETAILED DESCRIPTION

The particular values and configurations discussed in the following non-limiting examples can be varied, and are cited merely to illustrate one or more embodiments and are not intended to limit the scope thereof.

Example embodiments will now be described more fully hereinafter with reference to the accompanying drawings, in which illustrative embodiments are shown. The embodiments disclosed herein can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is contemplated that any embodiment discussed in this specification can be implemented with respect to any method, kit, reagent, or composition of the invention, and vice versa. Furthermore, compositions of the invention can be used to achieve methods of the invention.

It will be understood the principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context. All aspects of the various embodiments can be interchangeable with aspects disclosed in other embodiments, and/or can be incorporated in other embodiments.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope and concept of the invention as defined by the appended claims.

The embodiments disclosed herein are directed to cannulation systems and associated methods for cannulation. It should be appreciated that hemodialysis is a very common procedure where cannulation systems, as disclosed, may be used, but the cannulation systems described herein can also be used for other applications, including but not limited to, plasmapheresis, rapid peripheral/central vascular access in intensive care units and emergency departments, military applications, and the like. In an exemplary embodiment, an innovative cannulation system for use in hemodialysis, and associated procedures, is disclosed.

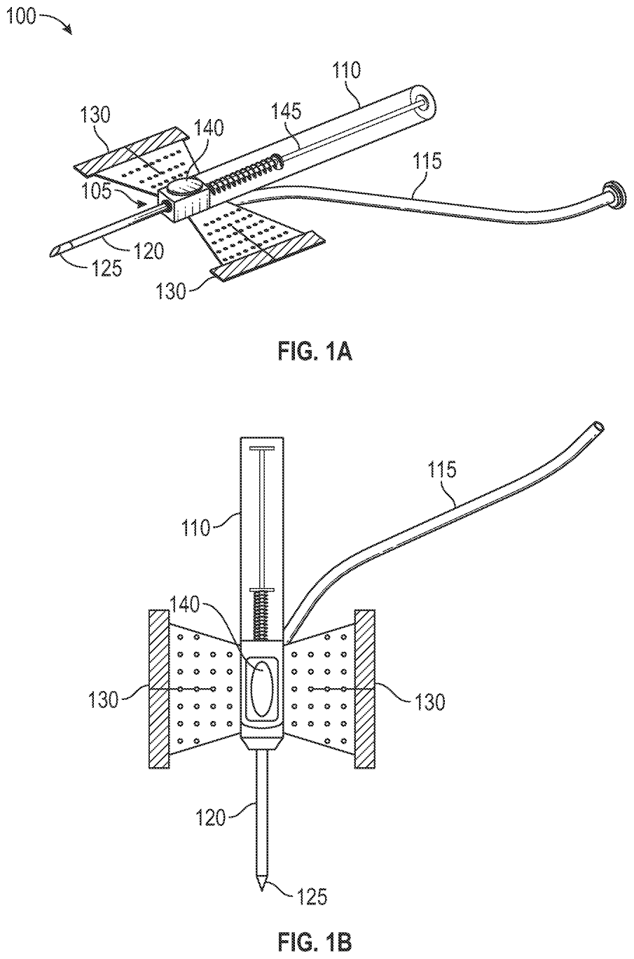

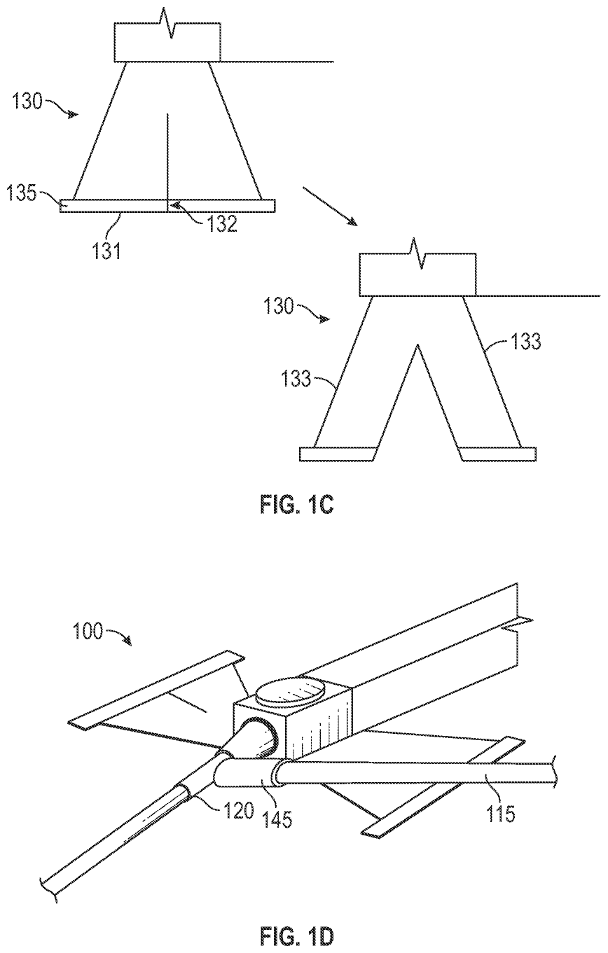

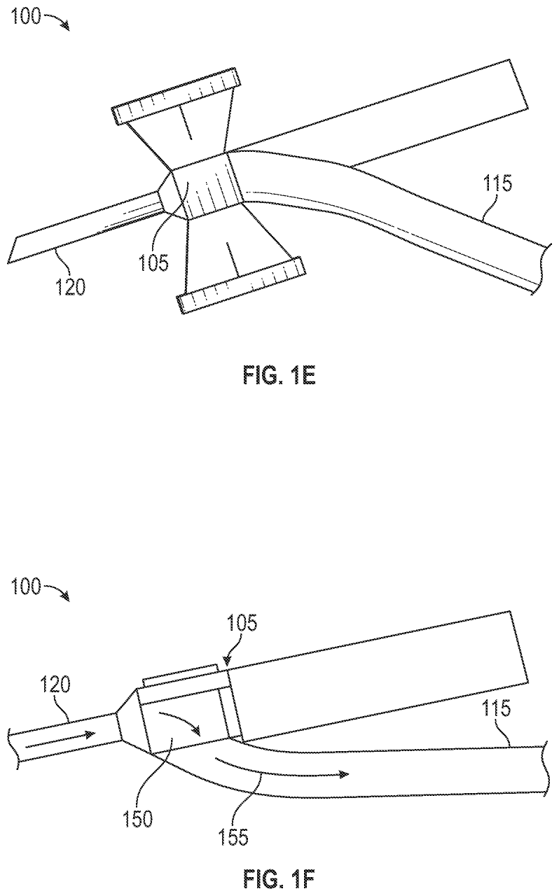

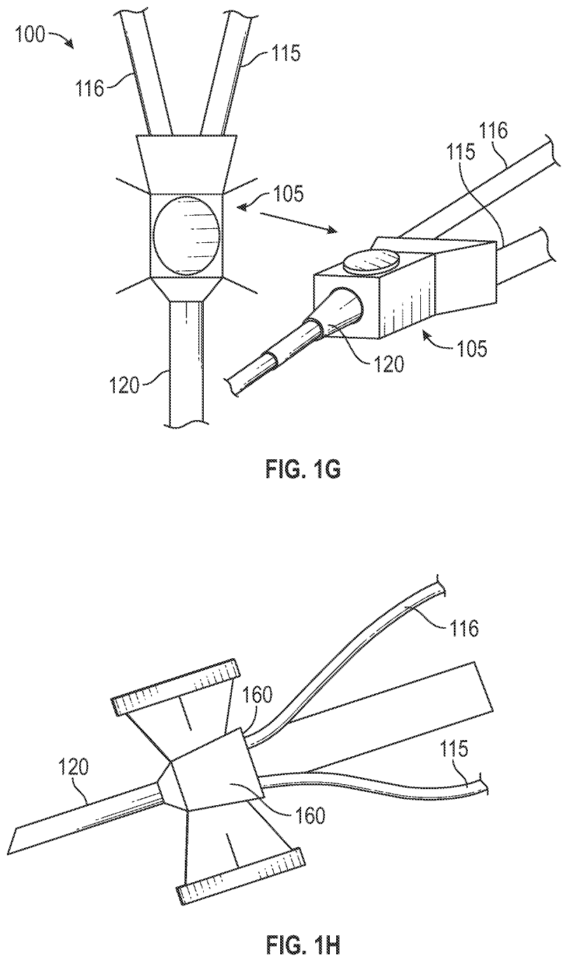

FIG. 1A illustrates the cannulation system 100. The cannulation system 100 generally includes a needle retraction assembly 105 a safety enclosure 110 and attached dialysis tubing 115. In addition, the system 100 has a cannula 120 which is coaxial with needle 125, when the needle 125 is deployed for insertion in a patient, and remains in the patient when the needle 125 is retracted, thereby providing vascular access.

Once inserted the cannula 120 can remain in the patient's vascular access for the duration of the prescribed treatment. Support tabs or wings 130 can be deployed to hold the assembly in place. Support wings 130 can connect to a button 140. They can be directly attached to the underside of button 140 and can attach via adhesive or other means. The support wings 130 can include adhesive 135 (on the underside of the wings 130) for attachment to the patient's skin. Before use, the adhesive 135 can be covered by a removable covering. In certain embodiments, the covering can be paper, although other material can be used. The top portion of the wings 130 need not be covered, only the bottom. The top portion of the wings 130 can have textured dots on them to improve grip.

FIG. 1C illustrates aspects of wings 130. As illustrated, the wings 130 can include tabs 131, and can be coated with an adhesive 135. In certain embodiments, a slit 132 can be formed along the center of the wings 130. The legs 133 of the wings 130 can be separated as illustrated in FIG. 1C to improve stability and adhesion.

FIG. 1B illustrates a top plan view of the cannulation system 100. The needle 125 can be retracted into the needle enclosure 110 with the operation of button 140 formed on the retraction assembly 105. The needle enclosure or safety enclosure 110 can be threaded at one end so that it can be twisted off to discard the used needle 125. From the detachment point, blood samples can be drawn for requested laboratory testing. Enclosure 110 can be threaded on the adjacent joining point with the retraction assembly 105. The safety enclosure 110 can have a hemostatic seal on the inside of the opening which will seal once the needle is retracted fully.

An aspect of the disclosed embodiments is that the cannula 120 is initially configured to have a diameter slightly larger than the needle 125. The cannula 120 can be configured as a sheath through which the needle 125 can fit. In certain embodiments, the cannula 120 can comprise a plastic cannula, although other materials can also be used, provided the cannula adequately surrounds the needle 125. In certain embodiments, the material can preferably be Polytetrafluoroethylene (PTFE). In other embodiments, silicone can be used. The catheter must be somewhat flexible. The overall apparatus will be sterilized prior to packaging and will remain sterile until opened by a provider.

Likewise, in certain embodiments the needle 125 can comprise a stainless steel needle, although other material choices can be used as necessary. The gauge and/or length of the needle 125 and cannula 120 assembly can be determined depending on the application.

FIG. 1D illustrates an embodiment of a dialysis catheter with a single lumen, illustrating how the tubing 115 can be connected to the system 100. In an embodiment, the tubing 115 can be connected to the cannula 120 via joint 145. This is an exemplary connection, but other connection arrangements can also be used.

For example, FIG. 1E provides a bottom view and FIG. 1F provides a side elevation view illustrating a single lumen HD catheter with the tubing 115 connecting below the button housing. In this embodiment, the tubing 115 can connect to a chamber 150 so that fluid can flow from the cannula 120 through the chamber 150 and into the tubing 115. Arrows 155 illustrate the flow of fluid through the system 100.

FIG. 1G and FIG. 1H illustrate a double lumen HD catheter with the tubing 115 and tubing 116 connecting below the button housing. In this embodiment, the tubing 115 and tubing 116 can connect to a chamber 160 so that fluid can flow from the cannula 120 through the chamber 150 and into the tubing 115 and the tubing 116.

FIG. 1I illustrates another embodiment of the system 100 comprising a triple lumen catheter. In this embodiment, a splitter 165 can fluidically connect to the cannula 120. The splitter 165 can include outputs for tubing 115, tubing 116, and tubing 117. In certain embodiments, the opening 170, opening 171, and opening 172, in the splitter 165 can be sized to fit different diameter tubing.

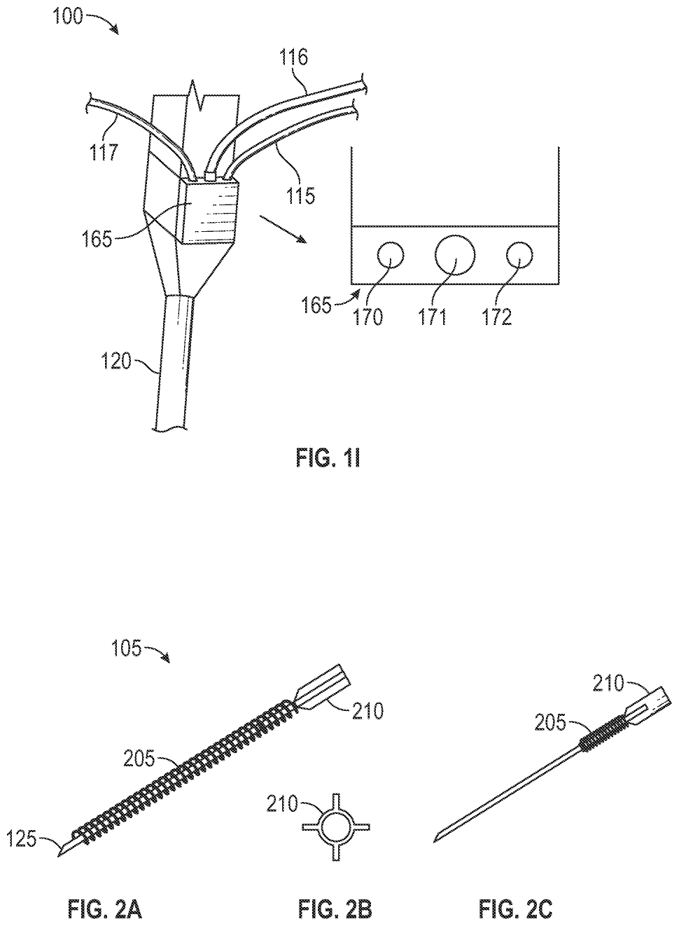

FIG. 2A illustrates the needle 125 in the deployed state, equipped with a spring 205 and spring stop 210. The spring 205 is used to retract the needle 125 through the cannula after it has been used for vascular access. The spring stop's 210 function is to direct the retracted needle 124 into the receptacle and maintain its center position.

FIG. 2B provides an exploded top view of the spring stop 210.

FIG. 2C illustrates the needle 125 with the spring 205 in the non-deployed state before it has been used to retract the needle 125 into the safety enclosure 110.

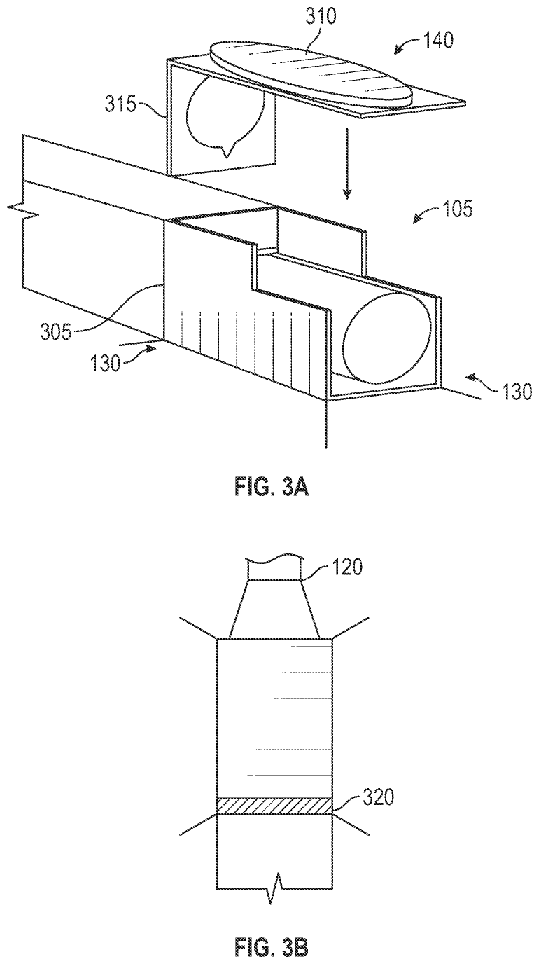

FIG. 3A and FIG. 3B illustrate aspects of the needle retraction assembly 140. The needle retraction assembly 140 can be attached to a plastic hub with the button 310 on the dorsal aspect of the cannulation system 100. When the button 310 is depressed, the retraction plate 315 slides into void 320, so that the spring 205 can retract the needle 125 into the adjacent needle chamber 110, leaving the cannula 120 inside of the blood vessel, which allows blood to be withdrawn or medication/therapeutics to be administered. In operation, a small ledge on the needle 125 is held by a notch on button 140. When the button 140 is depressed, the ledge will no longer be held back by the notch and from the spring's force, will be retracted back into the receptacle.

Once button 140 is deployed, the needle 125 immediately retracts into the safety enclosure 110. With the needle 125 retracted, blood can flow through the plastic cannula 120 into the button housing 305 and attached plastic tubing 115. The housing 305 can include a portion slotted out to show when the vascular space has been adequately accessed. Once the needle 125 is retracted, the initial hemostatic seal will prevent flow into the needle receptacle and divert flow into the adjoined tubing.

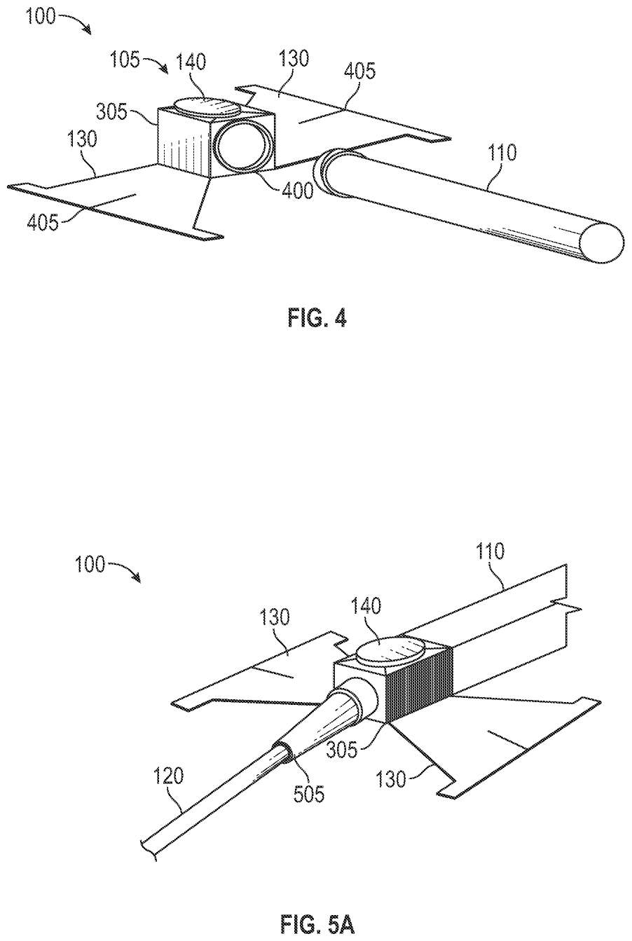

FIG. 4, illustrates aspects of the retraction assembly 105 and safety enclosure 110. The retraction assembly 105 can include the button housing 305, retraction button 140, as well as a connection port comprising Luer lock 400 which is part of the connection assembly. The safety enclosure 110 can be disengaged by twisting off the enclosure 110 in a clockwise direction.

The attached stability “wings” 130 can provide increased control of the cannulation system 100 during insertion. The stability wings 130 can also be used secure the system 100 to the patient by removing a paper backing on the dorsal aspect of the wings 130 exposing an adhesive 135. In certain embodiments, the wings 130 can be divided along a precut division 405, so that and the wings can be stretched to secure the device to the skin.

The wings 130 are an aspect of the cannulation system 100 that allows the patient to self-cannulate with one hand. Specifically, the wings 130 provide increased control and stability of the device allowing for increasing freedom to dialyze at home. To that end, the wings 130 can be configured of a rigid or semi-rigid material which is sufficiently stiff to hold for improved control and/or to be used as a guide.

FIG. 5A illustrates further aspects of the system 100 in accordance with the disclosed embodiments. In this embodiment, the plastic cannula 120 is illustrated with the needle 125 retracted (and therefore not visible). As illustrated, the cannula 120 can connect to a transparent or translucent hub 505 mounted to the button housing 305, which can also be transparent or translucent. The rear of the button housing 305 is connected to the transparent or translucent safety enclosure 110.

The flash chamber allows the user to see the flow of blood in order to verify that vascular access has been achieved. It is therefore an aspect of the embodiments, that the flash chamber be made of material that is see through. Similarly, the safety enclosure 110 can be configured with a transparent or translucent material so that the user can verify that the needle has been retracted and is safely inside the safety enclosure 100.

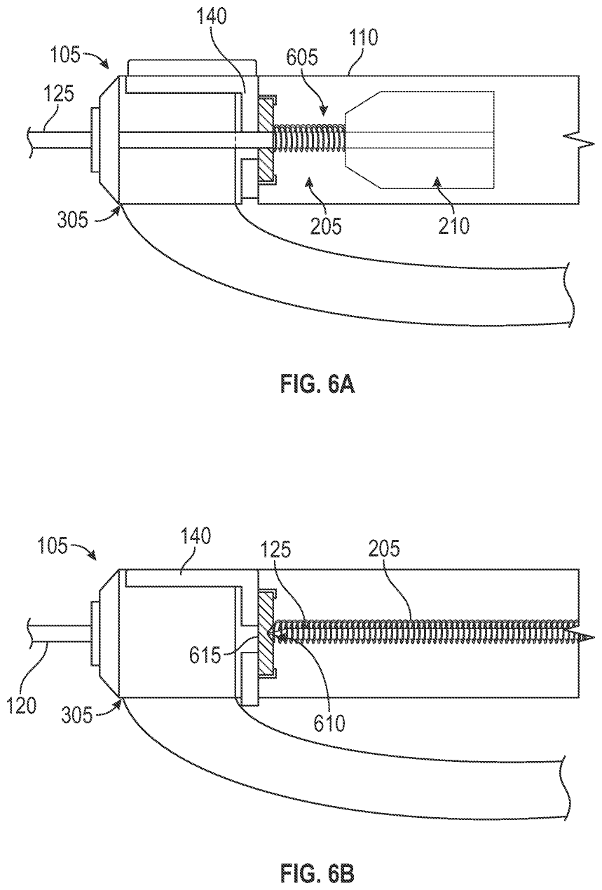

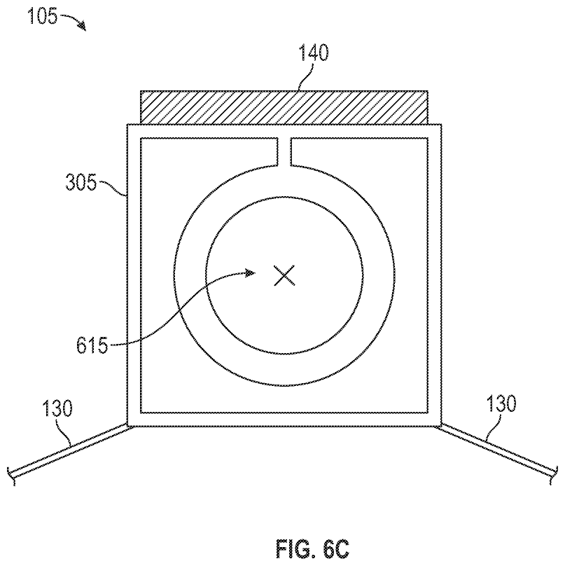

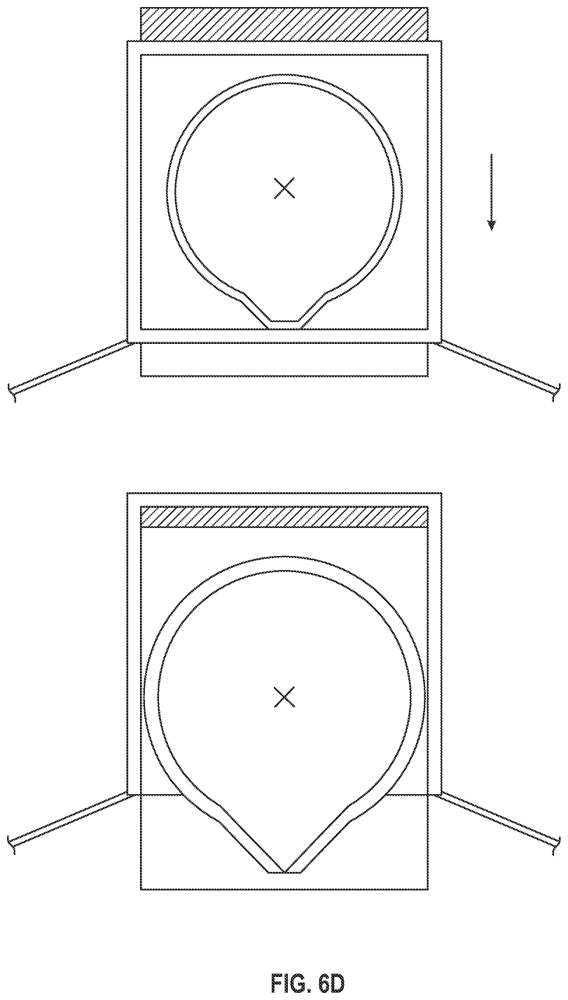

FIGS. 6A and 6B illustrate aspects of the needle retraction assembly 105. FIG. 6A illustrates the needle in a “non-deployed” position, meaning the needle 125 is extending out the front end of the needle retraction assembly 105 in the cannula 120. FIG. 6B illustrates the needle 125 retracted into the safety enclosure 110.

As illustrated in FIG. 6A, when the needle is in a non-deployed position, the spring 205 is compressed. The spring stop 210 can be connected to the distal end of the needle 125. The back end 605 of the spring 205 can be connected to the spring stop 210, while the front end 610 of the spring 205 can be pressing against the surface of the housing 305.

In FIG. 6B the button 140 has been operated and the needle is in the “deployed” position. The energy stored in the spring 205 has been released, forcing the spring stop 210 connected to the needle 125, backward into the safety enclosure 110. In certain embodiments, a hydrostatic/hemostatic seal 615 can be located immediately before the safety enclosure 110, as a part of the needle retraction assembly 105. The seal 615 can be made of rubber in certain embodiments, although other materials can also be used. The seal 615 prevents blood/IVF/therapeutics from passing between the retraction assembly 105 and the safety enclosure 110.

FIG. 6C illustrates a cross-sectional view of the needle retraction assembly 105. FIG. 6D illustrates the needle extraction assembly with the button depressed and not depressed.

It should be appreciated that if blood samples are to be retrieved or medications are to be administered, a syringe can be attached via the luer lock mechanism 400. Once the safety enclosure 110 is disengaged, the hydro/hemostatic rubber seal can be covered by use of antibacterial plastic lock (not shown).

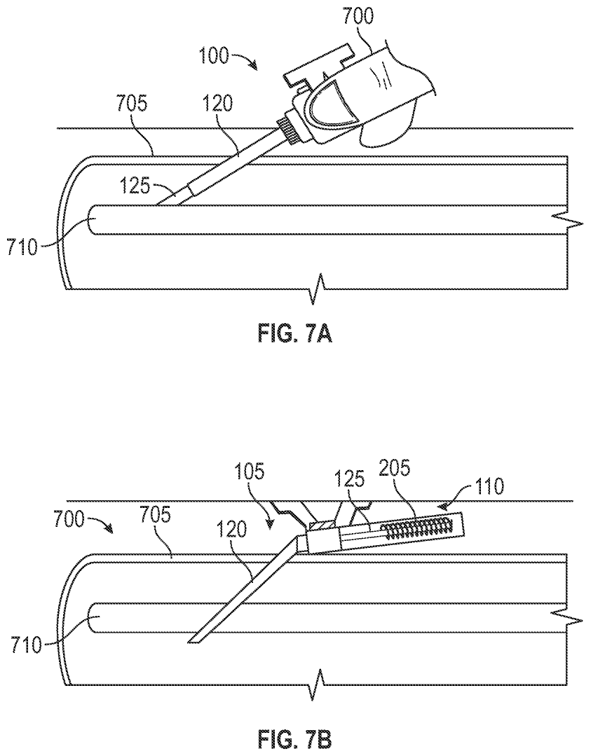

FIGS. 7A and 7B illustrate the system 100, used to provide vascular access. In FIG. 7A, a user 700 can insert the needle 125 and cannula 120 through the dermal layers 705 of a patient and into a vessel 710. After vascular access has been achieved, the needle 125 can be retracted as illustrated in FIG. 7B. As illustrated, the cannula 120 remains in the vessel 710, but the needle 125 is retracted into the safety enclosure 110.

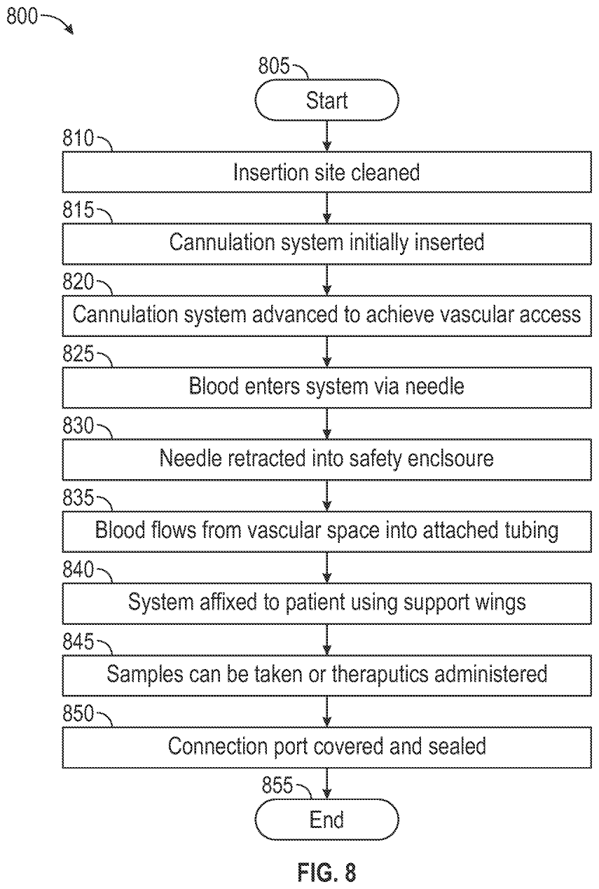

FIG. 8 illustrates a method 800 associated with the system 100 in accordance with the disclosed embodiments. The method begins at 805.

At step 810, the insertion site can be identified and properly cleaned with the aseptic technique. Next at step 815, the cannulation system can be initially inserted into the patient. In certain embodiments, this can be done either by the patient or by a trained medical provider. The cannulation system can be advanced using the support wings 130 for control at step 820. In this step, the needle and cannula are advanced together along a transdermal pathway at an angle suitable for insertion.

At step 825, blood will enter the system via needle. The blood will flow through the needle and enter flash chamber signifying a vascular space has been entered. Once the appropriate vascular space has been achieved, the needle retraction button can be operated at step 830 causing the needle to retract into the safety enclosure, which can be disengaged for further safety. Note, the cannula remains inside the vascular space.

At step 835, blood from the vascular space can proceed through the cannula into the attached tubing for further medical interventions which include but are not limited to hemodialysis, plasmapheresis, rapid blood product transfusion, and/or medications/therapeutic interventions may be administered (such as blood products or vasopressors).

At step 840 the adhesive on the posterior side of the support wings can be exposed by removal of covering material and optionally torn along the pre-cut slits, stretched, and finally secured to the adjacent skin.

If/when the safety enclosure is disengaged, blood samples can be retrieved via the Luer Lock connection and/or medications/therapeutics can be administered through said Luer Lock connection as illustrated at step 845. The method prevents blood from leaking through this connection due to hemostatic valve formed thereon. Finally, at step 850, the connection port can be covered and sealed by means of an antibacterial cap for later use, if warranted. The method ends at 850.

Based on the foregoing, it can be appreciated that a number of embodiments, preferred and alternative, are disclosed herein. In an embodiment, a system comprises a cannula, a needle, a needle retraction assembly, and a safety enclosure wherein the needle retraction assembly is configured to draw the needle out of the cannula and into the safety enclosure. In an embodiment, the needle is configured to be coaxial with the cannula. In an embodiment, the system further comprises at least one support wing attached to the needle retraction assembly. In an embodiment, the support wing further comprises an adhesive and a removable cover covering the adhesive. In an embodiment, the support wing further comprises a preformed slit along the support wing. In an embodiment, the safety enclosure is configured to be disengaged from the needle retraction assembly. In an embodiment, the system further comprises a connection port. In an embodiment, the connection port further comprises a Luer lock system configured on the needle retraction assembly. In an embodiment, the needle retraction assembly further comprises a spring engaged to the needle with a spring stop, a housing, and a button formed on the housing, the button used to release the spring.

In an embodiment, the cannulation system comprises a needle configured coaxially inside a cannula, a needle retraction assembly comprising a spring engaged to the needle with a spring stop and a button formed on the housing, the button used to release the spring and a safety enclosure wherein the needle retraction assembly is configured to draw the needle out of the cannula and into the safety enclosure when the button is operated. In an embodiment, the cannulation system further comprises at least one support wing attached to the needle retraction assembly. In an embodiment, the support wing further comprises an adhesive and a removable cover covering the adhesive. In an embodiment, the safety enclosure is configured to be disengaged from the needle retraction assembly. In an embodiment, the cannulation system further comprises a Luer lock system configured on the needle retraction assembly.

In another embodiment, a system comprises a needle configured coaxially inside a cannula, a needle retraction assembly, a safety enclosure wherein the needle retraction assembly is configured to draw the needle out of the cannula and into the safety enclosure, and tubing connected to the cannula. In an embodiment the system further comprises a joint connecting the tubing to the cannula. In an embodiment, the system further comprises a chamber connecting the cannula to the tubing, wherein fluid can flow from the cannula through the chamber into the tubing. In an embodiment, the system further comprises at least one support wing attached to the needle retraction assembly, the support wing further comprising: an adhesive, a removable cover covering the adhesive, and a preformed slit along the support wing. In an embodiment, the system further comprises a Luer lock system configured on the needle retraction assembly. In an embodiment, the needle retraction assembly further comprises a spring engaged to the needle with a spring stop, a housing, and a button formed on the housing, the button used to release the spring.

It should be appreciated that variations of the above-disclosed and other features and functions, or alternatives thereof, may be desirably combined into many other different systems or applications. It should be understood that various presently unforeseen or unanticipated alternatives, modifications, variations, or improvements therein may be subsequently made by those skilled in the art which are also intended to be encompassed by the following claims.

What is claimed is:

1. A system comprising:
a cannula;
a needle;
a needle retraction assembly; and
a safety enclosure wherein the needle retraction assembly is configured to draw the needle out of the cannula and into the safety enclosure.

2. The system of claim 1 wherein the needle is configured to be coaxial with the cannula.

3. The system of claim 1 further comprising:
at least one support wing attached to the needle retraction assembly.

4. The system of claim 3 wherein the support wing further comprises:
an adhesive; and
a removable cover covering the adhesive.

5. The system of claim 3 wherein the support wing further comprises:
a preformed slit along the support wing.

6. The system of claim 1 wherein the safety enclosure is configured to be disengaged from the needle retraction assembly.

7. The system of claim 1 further comprising:
a connection port.

8. The system of claim 5 wherein the connection port further comprises:
a Luer lock system configured on the needle retraction assembly.

9. The system of claim 1 wherein the needle retraction assembly further comprises:
a spring engaged to the needle with a spring stop;
a housing; and
a button formed on the housing, the button used to release the spring.

10. A cannulation system comprising:
a needle configured coaxially inside a cannula;
a needle retraction assembly comprising:
a spring engaged to the needle with a spring stop; and
a button formed on the housing, the button used to release the spring; and
a safety enclosure wherein the needle retraction assembly is configured to draw the needle out of the cannula and into the safety enclosure when the button is operated.

11. The cannulation system of claim 10 further comprising:
at least one support wing attached to the needle retraction assembly.

12. The cannulation system of claim 11 wherein the support wing further comprises:
an adhesive; and
a removable cover covering the adhesive.

13. The cannulation system of claim 10 wherein the safety enclosure is configured to be disengaged from the needle retraction assembly.

14. The cannulation system of claim 10 further comprising:
a Luer lock system configured on the needle retraction assembly.

15. A system comprising:
a needle configured coaxially inside a cannula;
a needle retraction assembly;
a safety enclosure wherein the needle retraction assembly is configured to draw the needle out of the cannula and into the safety enclosure; and
tubing connected to the cannula.

16. The system of claim 15 further comprising:
a joint connecting the tubing to the cannula.

17. The system of claim 15 further comprising:
a chamber connecting the cannula to the tubing, wherein fluid can flow from the cannula through the chamber into the tubing.

18. The system of claim 15 further comprising:
at least one support wing attached to the needle retraction assembly, the support wing further comprising:
an adhesive;
a removable cover covering the adhesive; and
a preformed slit along the support wing.

19. The system of claim 15 further comprising:
a Luer lock system configured on the needle retraction assembly.

20. The system of claim 15 wherein the needle retraction assembly further comprises:
a spring engaged to the needle with a spring stop;
a housing; and
a button formed on the housing, the button used to release the spring.
